(19) United States
(12) Patent Application Publication
(10) Pub. No.: US 2009/0088772 A1
(43) Pub. Date: Apr. 2, 2009

(54) FIBER OPTIC IN-SITU CHEMICAL ANALYSIS IN A ROBOTIC SURGICAL SYSTEM

(76) Inventor: Stephen J. Blumenkranz, Redwood City, CA (US)

Correspondence Address: Frank Nguyen;Intuitive Surgical, Building 101, 1266 Kifer Road, Sunnyvale, CA, 94086, US

(21) Appl. No.: 11/863,142

(22) Filed: Sep. 27, 2007

(51) Int. Cl. A61B 19/00

(52) U.S. Cl. 606/130

(57) ABSTRACT

In one embodiment, a surgical instrument is described, the instrument including a housing that operably interfaces with a manipulator arm of a robotic surgical system, a shaft including a lengthwise axis, a wrist joint operably coupled to the distal end of the shaft, an end portion operably coupled to the wrist joint, and an optical fiber having a first end operably coupled to the end portion and a second end operably coupled to a spectrophotometer. A system and method for using the surgical instrument are also described.

### TECHNICAL FIELD

The present invention is generally related to medical and/or robotic devices, systems, and methods.

### BACKGROUND

Minimally invasive medical techniques are intended to reduce the amount of extraneous tissue that is damaged during diagnostic or surgical procedures, thereby reducing patient recovery time, discomfort, and deleterious side effects. One effect of minimally invasive surgery, for example, may be reduced post-operative hospital recovery times. Because the average hospital stay for a standard surgery is typically significantly longer than the average stay for an analogous minimally invasive surgery, increased use of minimally invasive techniques could save millions of dollars in hospital costs each year. While many of the surgeries performed each year in the United States could potentially be performed in a minimally invasive manner, only a portion of the current surgeries use these advantageous techniques due to limitations in minimally invasive surgical instruments and the additional surgical training involved in mastering them.

Minimally invasive robotic surgical or telesurgical systems have been developed to increase a surgeon's dexterity and to avoid some of the limitations on traditional minimally invasive techniques. In telesurgery, the surgeon uses some form of remote control, e.g., a servomechanism or the like, to manipulate surgical instrument movements, rather than directly holding and moving the instruments by hand. In telesurgery systems, the surgeon can be provided with an image of the surgical site at the surgical workstation. While viewing a two or three dimensional image of the surgical site on a display, the surgeon performs the surgical procedures on the patient by manipulating master control devices, which in turn control motion of the servomechanically operated instruments.

In robotically-assisted surgery, the surgeon typically operates a master controller to control the motion of surgical instruments at the surgical site from a location that may be remote from the patient (e.g., across the operating room, in a different room, or a completely different building from the patient). The master controller usually includes one or more hand input devices, such as hand-held wrist gimbals, joysticks, exoskeletal gloves or the like, which are operatively coupled to the surgical instruments that are releasably coupled to a patient side surgical manipulator (“the slave”). The master controller controls the instruments' position, orientation, and articulation at the surgical site. The slave is an electro-mechanical assembly which includes a plurality of arms, joints, linkages, servo motors, etc. that are connected together to support and control the surgical instruments. In a surgical procedure, the surgical instruments (including an endoscope) may be introduced directly into an open surgical site or more typically through trocar sleeves into a body cavity. Depending on a surgical procedure, there are available a variety of surgical instruments, such as tissue graspers, needle drivers, electrosurgical cautery probes, etc., to perform various functions for the surgeon, e.g., holding or driving a needle, suturing, grasping a blood vessel, or dissecting, cauterizing or coagulating tissue.

A surgical manipulator assembly may be said to be divided into three main components that include a non-sterile drive and control component, a sterilizable end effector or surgical tool/instrument, and an intermediate connector component. The intermediate connector component includes mechanical elements for coupling the surgical tool with the drive and control component, and for transferring motion from the drive component to the surgical tool.

During surgery (or for pre-operative diagnosis) it may be desired to identify the chemical characteristics of the tissue being examined or the chemistry of liquids surrounding the tissue to determine the presence of a suspected pathology. For example, a tumor may be identified by the presence of natural chemicals markers or artificially administered chemical markers that target the diseased tissue. Typically, such analyses are done by taking a biopsy sample and sending the sample to a laboratory for analysis, which requires time, handling, and exposure to other environments before the analysis can be completed. The ability to identify the chemistry and pathology in situ and substantially in real time would greatly speed the decision making and the procedure in confident removal of diseased tissue.

### SUMMARY

The present invention utilizes optic fiber guided light with minimally invasive surgical instruments and a spectrophotometer to sample the spectral characteristics of tissue in-situ and substantially in real time.

In accordance with an embodiment of the present invention, a surgical instrument is provided, the instrument including a housing that operably interfaces with a manipulator arm of a robotic surgical system and a shaft including a lengthwise axis. A wrist joint is operably coupled to the distal end of the shaft and an end portion is operably coupled to the wrist joint. An optical fiber has a first end operably coupled to the end portion and a second end operably coupled to a spectrophotometer.

In accordance with another embodiment of the present invention, a robotic surgical system is provided, the system including a manipulator assembly including a manipulator arm, a surgical instrument as described above, and a spectrophotometer operably coupled to a second end of the optical fiber for optical emission analysis.

In accordance with yet another embodiment of the present invention, a method of in-situ chemical analysis during a robotic surgical procedure is provided, the method including providing a surgical instrument as described above and operably coupling the housing of the surgical instrument to a manipulator arm of a robotic surgical system. Light is transmitted through the optical fiber and then light is received in the spectrophotometer for analysis.

Advantageously, embodiments of the present invention provide for in situ and substantially real time analysis of a surgical area, thereby providing for reduced time and invasiveness of tissue pathology determination during surgery or pre-operative diagnostics. Other advantages of the invention are provided.

The scope of the invention is defined by the claims, which are incorporated into this section by reference. A more complete understanding of embodiments of the present invention will be afforded to those skilled in the art, as well as a realization of additional advantages thereof, by a consideration of the following detailed description of one or more embodiments. Reference will be made to the appended sheets of drawings that will first be described briefly.

### BRIEF DESCRIPTION OF THE DRAWINGS

FIG. 1 is a schematic plan view of a portion of an operating theater illustrating a robotic surgical system, including a master surgeon console or workstation for inputting a surgical procedure and a robotic manipulator system for robotically moving surgical instruments at a surgical site within a patient.

FIGS. 2A and 2B illustrate a perspective view and a front view, respectively, of an embodiment of a manipulator system, including positioning linkages or set up joints which allow a patient side robotic manipulator and/or an endoscope or camera robotic manipulator to be pre-configured for surgery.

FIGS. 3A-3B and 4A-4B are perspective views and respective side views of a manipulator including a telescopic insertion axis, instrument interface, and instrument in accordance with an embodiment of the present invention.

FIG. 5 is a perspective view of an example of a surgical instrument for use in the system of FIG. 1.

FIGS. 6A and 6B are perspective views of portions of another surgical instrument and a chemical analyzer.

FIG. 7 is a flowchart illustrating a method of in-situ chemical analysis using a robotic surgical instrument.

Embodiments of the present invention and their advantages are best understood by referring to the detailed description that follows. It should be appreciated that like reference numerals are used to identify like elements illustrated in one or more of the figures. It should also be appreciated that the figures may not be necessarily drawn to scale.

### DETAILED DESCRIPTION

The present invention generally provides an improved surgical instrument, system, and method that utilizes optic fibers with minimally invasive surgical instruments and a spectrophotometer to sample in situ and in real time the spectral characteristics of tissue and/or surrounding environments.

The present invention is particularly useful as part of a telerobotic surgical system that allows the surgeon to manipulate the surgical instruments through a servomechanism from/at a location remote from the patient. One example of a robotic surgical system is the da Vinci® S™ surgical system available from Intuitive Surgical, Inc. of Sunnyvale, Calif. A User's Guide for the da Vinci® S™ surgical system is available from Intuitive Surgical, Inc. and is incorporated by reference herein for all purposes.

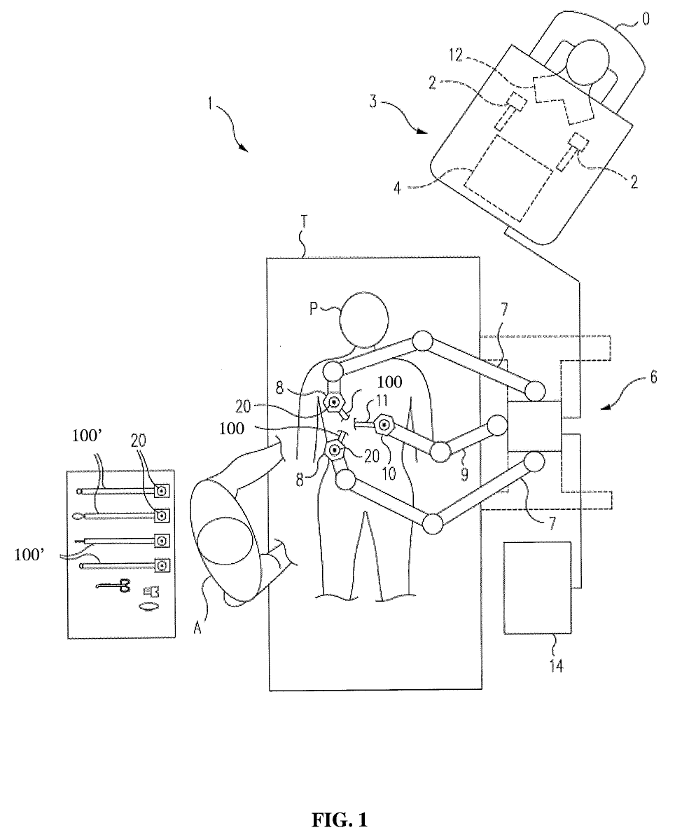

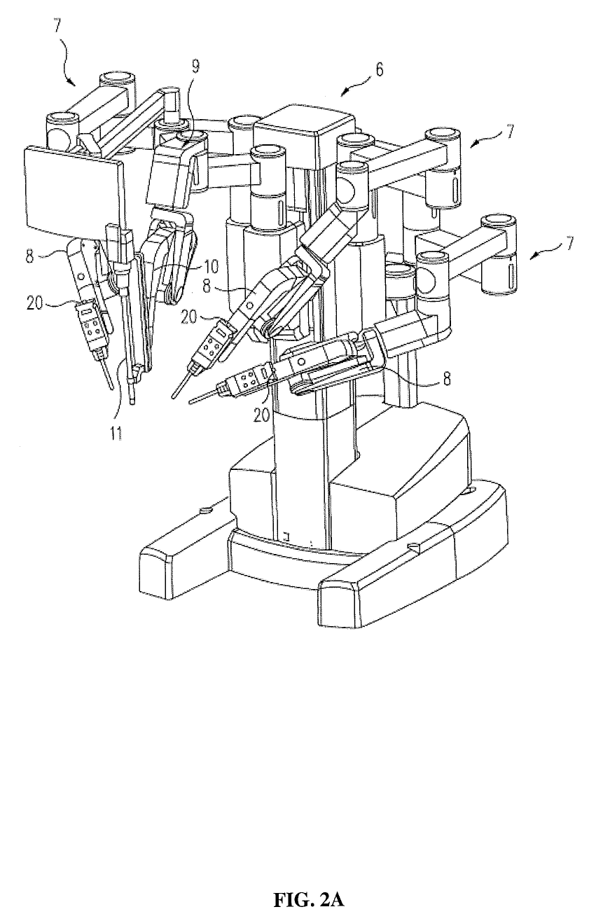

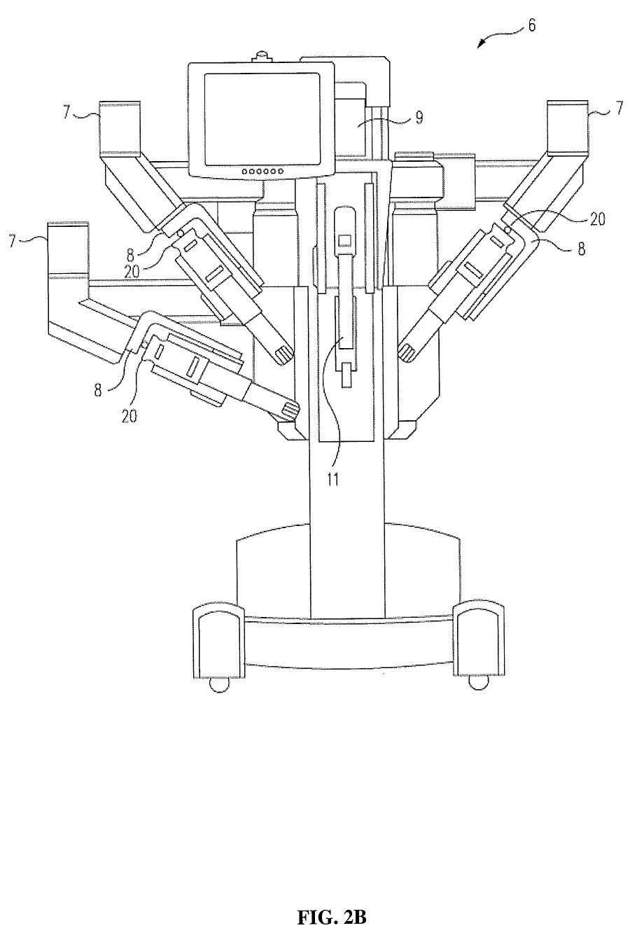

Referring now to FIGS. 1-2B, components of an example robotic surgical system 1 for performing minimally invasive robotic surgery are illustrated. System 1 is similar to that described in more detail in U.S. Pat. No. 6,246,200, the full disclosure of which is incorporated herein by reference. A system operator O (generally a surgeon) performs a minimally invasive surgical procedure on a patient P lying on an operating table T. The system operator O sees images presented by display 12 and manipulates one or more input devices or masters 2 at a surgeon's console 3. In response to the surgeon's input commands, a computer processor 4 of console 3 directs movement of surgical instruments or tools 100, effecting servomechanical movement of the instruments via a robotic patient-side manipulator system 6 (a cart-based system in this example) including joints, linkages, and manipulator arms each having a telescopic insertion axis in one example. In one embodiment, processor 4 correlates the movement of the end effectors of tools 100 so that the motions of the end effectors follow the movements of the input devices in the hands of the system operator O.

Processor 4 will typically include data processing hardware and software, with the software typically comprising machine-readable code. The machine-readable code will embody software programming instructions to implement some or all of the methods described herein. While processor 4 is shown as a single block in the simplified schematic of FIG. 1, the processor may comprise a number of data processing circuits, with at least a portion of the processing optionally being performed adjacent an input device, a portion being performed adjacent a manipulator, and the like. Any of a wide variety of centralized or distributed data processing architectures may be employed. Similarly, the programming code may be implemented as a number of separate programs or subroutines, or may be integrated into a number of other aspects of the robotic systems described herein.

In one example, manipulator system 6 includes at least four robotic manipulator assemblies. Three linkages 7 (mounted at the sides of the cart in this example) support and position manipulators 8 with linkages 7 in general supporting a base of the manipulators 8 at a fixed location during at least a portion of the surgical procedure. Manipulators 8 move surgical tools 100 for robotic manipulation of tissues. One additional linkage 9 (mounted at the center of the cart in this example) supports and positions manipulator 10 which controls the motion of an endoscope/camera probe 11 to capture an image (preferably stereoscopic) of the internal surgical site. The fixable portion of positioning linkages 7, 9 of the patient-side system is sometimes referred to herein as a “set-up arm”.

In one example, the image of the internal surgical site is shown to operator O by a stereoscopic display 12 in surgeon's console 3. The internal surgical site is simultaneously shown to assistant A by an assistance display 14.

Assistant A assists in pre-positioning manipulator assemblies 8 and 10 relative to patient P using set-up linkage arms 7, 9; in swapping tools 100 from one or more of the surgical manipulators for alternative surgical tools or instruments 100′; in operating related non-robotic medical instruments and equipment; in manually moving a manipulator assembly so that the associated tool accesses the internal surgical site through a different aperture, and the like.

In general terms, the linkages 7, 9 are used primarily during set-up of patient-side system 6, and typically remain in a fixed configuration during at least a portion of a surgical procedure. Manipulators 8, 10 each comprise a driven linkage which is actively articulated under the direction of surgeon's console 3. Although one or more of the joints of the set-up arm may optionally be driven and robotically controlled, at least some of the set-up arm joints may be configured for manual positioning by assistant A.

As can be seen in FIGS. 1 and 2A-2B, indicators 20 may be disposed on a manipulator assembly. In this embodiment, indicators 20 are disposed on manipulators 8 near the interface between the manipulators and their mounted tools 100. In alternative embodiments, indicators 20 may instead be disposed on set-up joints 7, 9, on tools 100, elsewhere on manipulators 8, 10, or the like. An example of an indicator is disclosed in U.S. patent application Ser. No. 11/556,484, filed Nov. 3, 2006, the full disclosure of which is incorporated by reference herein for all purposes.

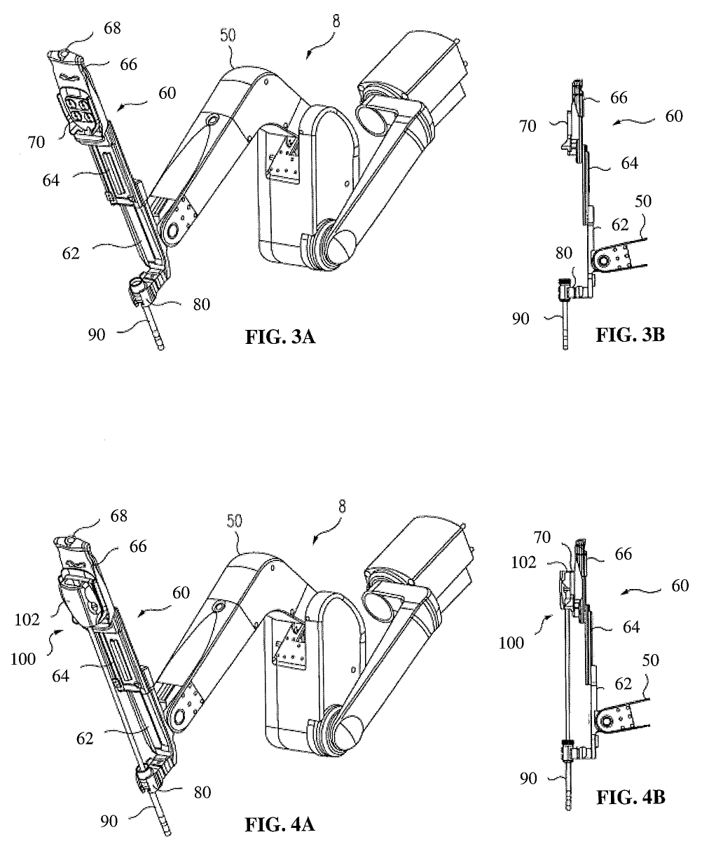

Referring now to FIGS. 3A-3B and 4A-4B, perspective views and respective side views of manipulator 8 including a manipulator arm 50 and telescopic insertion axis 60 operably coupled to a distal end of arm 50 are illustrated in accordance with an embodiment of the present invention. Telescopic insertion axis 60 includes a first link or base link 62, a second link or idler link 64 operably coupled to base link 62, and a third link or carriage link 66 operably coupled to idler link 64. Some of the manipulators include a telescopic insertion axis 60 in accordance with an embodiment of the present invention, although in other embodiments, all of the manipulators may include a telescopic insertion axis 60. Telescopic insertion axis 60 allows for movement of mounted tool or instrument 100, via three operably coupled links, with improved stiffness and strength compared to previous designs, a larger range of motion, and improved dynamic performance and visibility proximate the surgical field for system users (in addition to other advantages), as is described in greater detail in pending U.S. patent application Ser. No. 11/613,800, filed Dec. 20, 2006, which is incorporated by reference herein for all purposes.

Base link 62 is operably coupled to a distal end of manipulator arm 50, and in one example has an accessory clamp 80 attached to a distal end of base link 62. An accessory 90, such as a cannula, may be mounted onto accessory clamp 80. An example of applicable accessory clamps and accessories are disclosed in pending U.S. patent application Ser. No. 11/240,087, filed Sep. 30, 2005, the full disclosure of which is incorporated by reference herein for all purposes. An example of applicable sterile adaptors and instrument housings are disclosed in U.S. patent application Ser. No. 11/314,040, filed Dec. 20, 2005 and in U.S. patent application Ser. No. 11/395,418, filed Mar. 31, 2006, the full disclosures of which are incorporated by reference herein for all purposes.

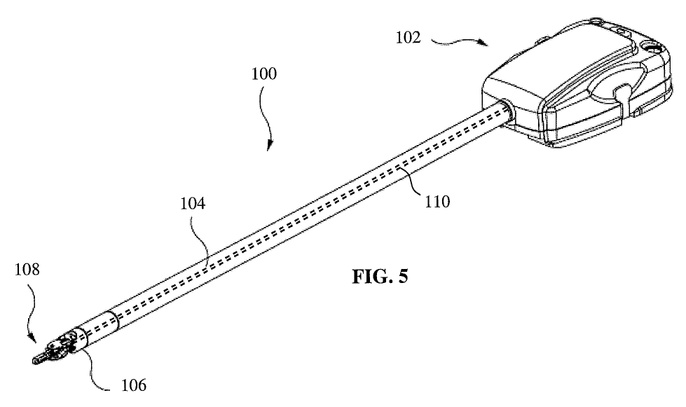

Carriage link 66 includes an instrument interface for operably coupling (e.g., electrically and/or physically) to an instrument sterile adaptor (ISA) 70, which is capable of operably coupling (e.g., electrically and/or physically) to a housing of an instrument (e.g., housing 102 of FIGS. 4 and 5), and controls the depth of the instrument inside a patient. In one embodiment, the sterile adaptor is integrated with a drape that may be draped over the robotic surgical system, and in particular the manipulator system, to establish a sterile barrier between the non-sterile PSM arms and the sterile field of the surgical procedure. An example of an applicable drape and adaptor is disclosed in pending U.S. patent application Ser. No. 11/240,113, filed Sep. 30, 2005, the full disclosure of which is incorporated by reference herein for all purposes. An example of an instrument interface is disclosed in pending U.S. patent application Ser. No. 11/613,695, filed Dec. 20, 2006, the full disclosure of which is incorporated by reference herein for all purposes.

Idler link 64 is movably coupled between base link 62 and carriage link 66 to allow the links 62, 64, and 66 to move relative to one another along a lengthwise axis in a telescoping fashion. In one embodiment, base link 62 has a narrower form factor than idler link 64, and idler link 64 has a narrower form factor than carriage link 66, thus providing for greater visibility near the surgical field.

For convenience, a manipulator such as manipulator 8 that is supporting a surgical tool used to manipulate tissues is sometimes referred to as a patient-side manipulator (PSM), while a manipulator 10 which controls an image capture or data acquisition device such as endoscope 11 may be referred to as an endoscope-camera manipulator (ECM). The manipulators may optionally actuate, maneuver and control a wide variety of instruments or tools, image capture devices, and the like which are useful for surgery.

Instruments 100 and endoscope 11 may be manually positioned when setting up for a surgical procedure, when reconfiguring the manipulator system 6 for a different phase of a surgical procedure, when removing and replacing an instrument with an alternate instrument 100′, and the like. During such manual reconfiguring of the manipulator assembly by assistant A, the manipulator assembly may be placed in a different mode than is used during master/slave telesurgery, with the manually repositionable mode sometimes being referred to as a clutch mode. The manipulator assembly may change between the tissue manipulation mode and the clutch mode in response to an input such as pushing a button or switch on manipulator 8 (e.g., a clutch button/switch 68 in FIGS. 3A-3B and 4A-4B), or some other component to the manipulator assembly, thereby allowing assistant A to change the manipulator mode.

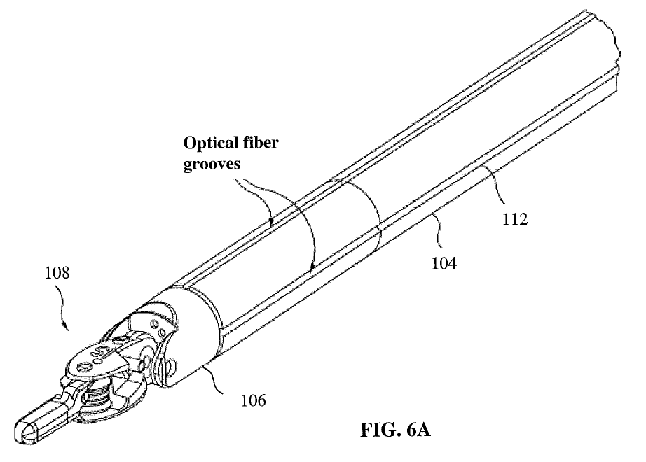

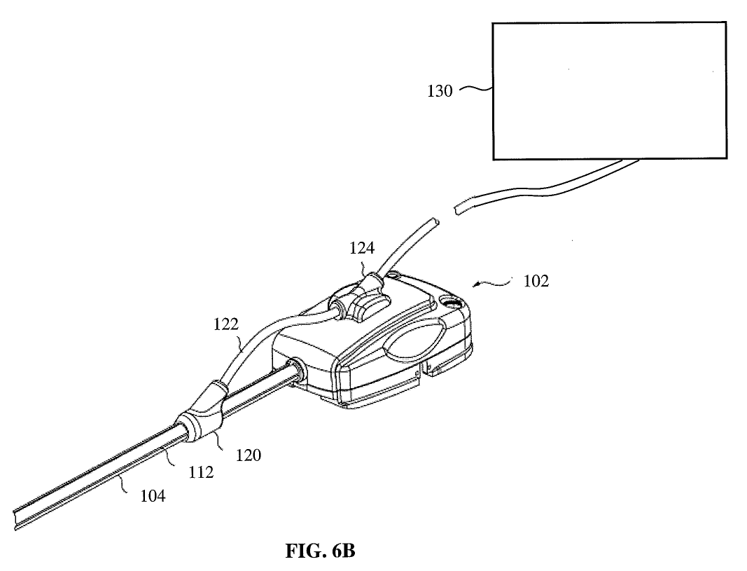

Referring now to FIGS. 5 and 6A-6B, FIG. 5 illustrates a perspective view of a surgical tool or instrument 100 in accordance with an embodiment of the present invention, and FIGS. 6A-6B illustrate views of portions of a surgical tool in accordance with another embodiment of the present invention. Tool 100 has a proximal housing 102 which interfaces with a tool holder or instrument interface of the manipulator, generally via a quick release mounting engagement through a sterile adapter or interface 70 (FIGS. 3A-3B), an example of which is disclosed in U.S. patent application Ser. No. 11/314,040, filed Dec. 20, 2005, and U.S. patent application Ser. No. 11/395,418, filed Mar. 31, 2006, which are incorporated by reference herein for all purposes. Tool 100 includes an elongated shaft 104 supporting an end effector 108 relative to proximal housing 102. Proximal housing 102 accepts and transmits drive signals and drive motion between the manipulator 8 and the end effector 108. An articulated wrist 106 may provide two degrees of freedom of motion between end effector 108 and shaft 104, and the shaft may be rotatable relative to proximal housing 102 about the lengthwise axis of the shaft so as to provide the end effector 108 with three orientational degrees of freedom within the patient's body.

The surgical tool may include a variety of articulated end effectors, such as jaws, scissors, graspers, needle holders, micro-dissectors, staple appliers, tackers, suction irrigation tools, and clip appliers, that may be driven by wire links, eccentric cams, push-rods, or other mechanisms. In addition, the surgical tool may comprise a non-articulated instrument, such as cutting blades, probes, irrigators, catheters, tubes, or suction devices. Alternatively, the surgical tool may comprise an electrosurgical probe for ablating, resecting, cutting or coagulating tissue. Examples of applicable adaptors, tools or instruments, and accessories are described in U.S. Pat. Nos. 6,331,181, 6,491,701, and 6,770,081, the full disclosures of which are incorporated by reference herein for all purposes.

In one embodiment, instrument 100 includes one or more optical fibers 110 transmitting light to and/or from the instrument tip/end effector or near the tip/end effector and operates in conjunction with a suitable analyzer 130, such as a Fourier transform infrared (FTIR) or ultraviolet-visible light (UV-VIS) spectrophotometer, capable of providing optical emission spectra for identifying the chemicals present in the optical path (e.g., transmissive or reflective). Optical emission spectroscopy allows for light emitted by a process to be analyzed for detection of certain chemical species because each kind of molecule or atom has a characteristic optical emission at specific wavelengths. The light may be in a suitable wavelength for spectral identification or analysis of tissue chemistry (e.g., surface chemistry on suspected tumors or cells, or binding agents, such as antibodies, drugs, or dye chemicals (markers), for binding to target cells of interest, such as tumor cells). For example, infrared light may be used to identify characteristic molecular bonds in organic bio-chemicals such as those that may be present in tissue. In another example, an FTIR spectrophotometer may provide an analytical result based on a plurality of averaged FTIR scans and a database search of candidate chemicals may be used to correlate the scans to chemicals.

In one embodiment, a single optic fiber may be used to examine reflected light as illustrated in FIG. 5. The optic fiber may be used for transmitting and receiving photons and is provided to both deliver excitation light at or near the instrument tip and to collect and transmit the scattered photons to the analyzer, such as a spectrophotometer. In other words, light is transmitted to the tissue or internal body fluid of interest and scattered spectrally altered light reenters the fiber and is transmitted to the spectrophotometer for analysis. In one example, light may be transmitted through an end or tip of the optic fiber. In a second example, light may be transmitted through an exposed or nearly exposed core of the optic fiber (i.e., light may be transmitted via exposed side surfaces of the optic fiber core) as in a fiber optic evanescent wave (FEW) sensor. The single optic fiber may be routed through the instrument shaft (e.g., via a center lumen), through or along the wrist, and to the end effector, or along a groove on an exterior surface of the instrument shaft, through or along the wrist, and to the end effector.

In another embodiment, two optic fibers may be used to enable transmissive analysis. Two fibers may be routed at a fixed gap or may be routed to opposite facing jaws of an end effector that may close over target tissue to create a transmissive path through the tissue to be analyzed. Light may be provided through one optic fiber, transmitted through the tissue, and emitted light received at the second optic fiber. The optic fibers may be routed through the instrument shaft (e.g., via a center lumen), through the wrist (e.g., via a center lumen), and to the end effector, or along grooves 112 on an exterior surface of the instrument shaft, through or along the wrist, and to the end effector, as illustrated in FIG. 6A. In a simple embodiment, the end effector may include a tube through which the optic fiber is utilized for chemical analysis.

Analyzer 130 may in one example be a spectrophotometer or any applicable optical instrument for measuring properties of light over a portion of the electromagnetic spectrum. Analyzer 130 may be used to select or filter certain frequencies from the optical emissions/reflections that are to be measured and can measure the intensity of the optical emissions/reflections. In one example, received light diffracts off a diffraction grating and is dispersed into its components. The dispersed light falls onto a detector which measures the light intensity. The result of this is a measurement of light intensity as a function of wavelength. The measured variable may include but is not limited to light intensity, polarization state, and other properties. The independent variable may include but is not limited to wavelength of the light, wavenumber, and electron volts. The detector of the spectrophotometer may be one of a focal plane array, a charge-coupled device (CCD), and a photodetector array, for measuring and recording the light spectrums based upon the received scattered photons from the tissue or solution of interest. The spectrophotometer may also transform an incoming time-domain waveform into a frequency-domain (or related) spectrum, or generally a sequence of such spectra. Other analyzers and spectrophotometers are also applicable.

In one embodiment, analyzer 130 may be mounted on the manipulator, or elsewhere in the surgical system or in the operating room separate from the manipulator, which could require routing of the optical fiber(s) across the sterile boundary.

A computer including a user interface may be operably coupled to analyzer 130 for inputting of parameters such as for calibration of the instrument's spectroscopy functions, selection of wavelength range, and/or diagnostic testing (e.g., proper functionality of optical fiber(s)). The computer may include a variety of typical processors and may include a general or special purpose processor, with network capabilities. In one example, the computer comprises a CPU, a memory, and a network adapter, which are interconnected by a bus. Other conventional means, such as a display, a keyboard, a printer, a bulk storage device, and a ROM, may also be connected to the bus. In one embodiment, processor 4 (FIG. 1) of the robotic surgical system may incorporate the functionality of a computer and user interface operably coupled to analyzer 130. In another embodiment, the computer and user interface as described above may be integrated within analyzer 130.

A light source for an optical fiber may include an LED, an SLD (Super Luminescent Diode), an incandescent light source, or a laser. In one embodiment, the light source for the apparatus is a light source of bandwidth appropriate for the procedure or detection of chemical species of interest and may range from near-IR to broadband. In one example, the light source bandwidth and optical fiber doping may be tailored for the procedure or chemical species of interest.

For all of the methods and apparatus mentioned above, it may be advantageous to use a calibration process for the spectrophotometer readings. This calibration may be done by using standards (e.g., standards of signals from known solutions or tissue chemistry) in one example prior to use. In any calibration method, the calibration data may be programmed into an integrated circuit embedded in the instrument or the manipulator so that the surgical system using the individual instrument can correctly identify and apply correction factors (e.g., standard baseline signals for subtraction from in situ signals) while the instrument is in use.

Optical fibers embedded in the instrument shaft preferably should exit the shaft near the proximal end of the instrument in a way that does not impede rotation of the shaft relative to the instrument housing/carriage while preserving the physical integrity of the fiber.

Referring now in particular to FIGS. 6A and 6B, in accordance with an embodiment of the present invention, optical fiber(s) may be embedded in shallow grooves 112 just below the shaft 104 surface near the instrument shaft distal tip just behind the wrist clevis, and then epoxied or otherwise potted into place. Grooves 112 may lead back toward the proximal end of the instrument which includes the motion inputs and wrist cable actuator mechanism (the “housing”) 102. Grooves 112 may be formed in the shaft during the initial pultrusion process or the grooves may be machined after shaft production. At a point near the proximal mechanism or housing, the fibers may be routed out of the grooves at a gentle angle and bundled through a strain relief 120 into a protective flexible sheath 122 which would carry the optical fibers to a strain relieved anchor point 124 on the top cover of the mechanism housing 102. The strain relief 120, flexible sheath 122, and anchor point 124 should have sufficient length and flexibility to permit safe repeated flexing and torsion as the instrument shaft 104 is rotated and thereby provides a strain relief service loop for safe and operable instrument movement.

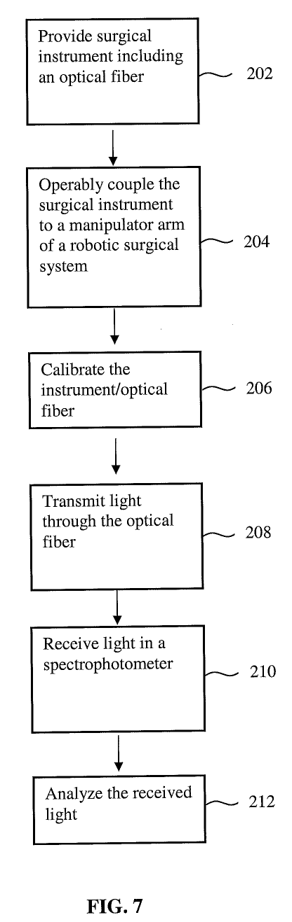

Referring now to FIG. 7, a flowchart is shown illustrating a method of in-situ chemical analysis using a robotic surgical instrument as described above. At step 202, a surgical instrument including an optical fiber such as that described above is provided. At step 204, the surgical instrument is operably coupled to a manipulator arm of a robotic surgical system, in one example as described above. Optionally, at step 206, optical emissions from the optical fiber are calibrated. At step 208, light from a light source is transmitted through the optical fiber of the surgical instrument, and at step 210, light is received at a spectrophotometer such as that described above. At step 212, the received light may be analyzed by various methods, such as ultraviolet-visible light spectroscopy, Fourier transform infrared (FTIR) spectroscopy, and Raman spectroscopy. A plurality of optical emission scans may also be averaged and correlated to chemicals.

Advantageously, embodiments of the present invention provide for real-time and in-situ analysis of a surgical area and/or tissue of interest, thereby providing for real-time analysis (versus laboratory analysis off-site or outside of the operating room) and reduced invasiveness of tissue pathology determination during surgery or pre-operative diagnostics.

Embodiments described above illustrate but do not limit the invention. It should also be understood that numerous modifications and variations are possible in accordance with the principles of the present invention. Accordingly, the scope of the invention is defined only by the following claims.

We claim:

1. A surgical instrument, comprising:
a housing that operably interfaces with a manipulator arm of a robotic surgical system;
a shaft including a lengthwise axis;
a wrist joint operably coupled to the distal end of the shaft;
an end portion operably coupled to the wrist joint; and
an optical fiber having a first end operably coupled to the end portion and a second end operably coupled to a spectrophotometer.

2. The instrument of claim 1, wherein the shaft includes a groove along the lengthwise axis of the shaft for receiving the optical fiber.

3. The instrument of claim 1, further comprising a second optical fiber operably coupled to the end portion and the spectrophotometer capable of transmissive analysis in conjunction with the optical fiber.

4. The instrument of claim 3, wherein the optical fiber and the second optical fiber are each routed to one of two jaws.

5. The instrument of claim 1, wherein the end portion of the surgical instrument is selected from the group consisting of jaws, scissors, graspers, needle holders, micro-dissectors, staple appliers, tackers, suction irrigation tools, clip appliers, cutting blades, irrigators, catheters, tubes, and suction devices.

6. The instrument of claim 1, wherein the housing interfaces with a sterile adaptor of a sterile drape covering the manipulator arm.

7. The instrument of claim 1, further comprising a rotatable strain relief service loop operably coupled to a proximal end of the shaft for routing at least one optical fiber off the shaft at an angle from the lengthwise axis of the shaft.

8. The instrument of claim 1, further comprising an integrated circuit storing calibration data for optical emissions.

9. A robotic surgical system, comprising:
a manipulator assembly including a manipulator arm;
a surgical instrument operably coupled to the manipulator arm, the surgical instrument including:
a housing that operably interfaces with the manipulator arm;
a shaft including a lengthwise axis;
a wrist joint operably coupled to the distal end of the shaft;
an end portion operably coupled to the wrist joint; and
an optical fiber having a first end operably coupled to the end portion; and
a spectrophotometer operably coupled to a second end of the optical fiber.

10. The system of claim 9, wherein the surgical instrument further includes a second optical fiber operably coupled to the end portion and the spectrophotometer capable of transmissive analysis in conjunction with the optical fiber.

11. The system of claim 9, wherein the end portion of the surgical instrument is selected from the group consisting of jaws, scissors, graspers, needle holders, micro-dissectors, staple appliers, tackers, suction irrigation tools, clip appliers, cutting blades, irrigators, catheters, tubes, and suction devices.

12. The system of claim 9, wherein the housing interfaces with a sterile adaptor of a sterile drape covering the manipulator arm.

13. The system of claim 9, wherein the surgical instrument further comprises a rotatable strain relief service loop operably coupled to a proximal end of the shaft for routing at least one optical fiber off the shaft at an angle from the lengthwise axis of the shaft.

14. The system of claim 9, wherein the spectrophotometer is capable of performing ultraviolet-visible light spectroscopy, Fourier transform infrared spectroscopy, or Raman spectroscopy.

15. A method of in-situ chemical analysis using a robotic surgical instrument including a housing that operably interfaces with a manipulator arm, a shaft including a lengthwise axis, a wrist joint operably coupled to the distal end of the shaft, an end portion operably coupled to the wrist joint, and an optical fiber having a first end operably coupled to the end portion and a second end operably coupled to a spectrophotometer, the method comprising:
operably coupling the housing of the surgical instrument to a manipulator arm of a robotic surgical system;
transmitting light through the optical fiber;
receiving light in the spectrophotometer; and
analyzing the received light.

16. The method of claim 15, wherein analyzing the received light includes one of ultraviolet-visible light spectroscopy, Fourier transform infrared spectroscopy, and Raman spectroscopy.

17. The method of claim 15, further comprising receiving reflected or transmitted light in the optical fiber.

18. The method of claim 15, further comprising averaging a plurality of optical emission scans and correlating chemicals to the optical emission scans.

19. The method of claim 15, further comprising transmitting light through a plurality of optical fibers.

20. The method of claim 15, further comprising calibrating optical emissions from the optical fiber.
